(19) United States
(12) Patent Application Publication
(10) Pub. No.: US 2014/0051802 A1
(43) Pub. Date: Feb. 20, 2014

(54) HIGH PURITY BISPHENOL-A AND POLYCARBONATE MATERIALS PREPARED THEREFROM

(71) Applicants: Johannes De Brouwer, Oisterwijk (NL); Paulus Johannes Maria Eijsbouts, Nieuwkijk (NL)

(72) Inventors: Johannes De Brouwer, Oisterwijk (NL); Paulus Johannes Maria Eijsbouts, Nieuwkijk (NL)

(73) Assignee: SABIC INNOVATIVE PLASTICS IP B.V., Bergen Op Zoom (NL)

(21) Appl. No.: 13/806,040

(22) PCT Filed: May 2, 2012
(86) PCT No.: PCT/IB2012/052198

§ 371 Date: Mar. 11, 2013

Related U.S. Application Data
(63) Continuation-in-part of application No. 13/099,032, filed on May 2, 2011.
Application No. 13/099,032 is a continuation-in-part of application No. 13/099,026, filed on May 2, 2011.
(60) Provisional application No. 61/618,351, filed on Mar. 30, 2012.

(51) Int. Cl. C08G 64/06; C08G 77/14; C07C 39/16

(52) U.S. Cl. CPC C08G 64/06; C07C 39/16; C08G 77/14
USPC 524/611; 568/723; 528/219; 528/29; 528/176; 525/534

(57) ABSTRACT

Methods for performing a condensation reaction are disclosed. Specifically, various methods for the production of highly-pure bisphenol-A are disclosed in which an attached promoter ion exchange resin catalyst system is combined with a solvent crystallization step.

### TECHNICAL FIELD

The present disclosure relates to catalyst systems, and specifically to promoter ion exchange resin catalyst systems.

### TECHNICAL BACKGROUND

Many conventional condensation reactions utilize inorganic acid catalysts, such as sulfuric acid or hydrochloric acid. Use of such inorganic acid catalysts can result in the formation of undesirable byproducts that must be separated from the reaction stream. Ion exchange resin catalyst systems can also be used, but the inherent low acid concentration can require the use of a promoter or rate accelerator.

When used as part of the catalyst system, reaction promoters can improve reaction rate and selectivity. In the case of the condensation of phenol and ketone to form bisphenol-A (BPA), reaction promoters can improve selectivity for the desired para-para BPA isomer.

Reaction promoters can be used as bulk promoters, where the promoter is present as an unattached molecule in the reaction medium, or as an attached promoter, where the promoter is attached to a sulphonic acidic portion of the catalyst system.

In the synthesis of BPA, the use of 3-mercaptopropionic acid (3-MPA) can produce a significant quantity of the less desirable o,p-BPA isomer, as opposed to the preferred p,p-BPA isomer.

While much effort has been applied to the development and use of bulk and attached promoter systems, a need still exists for a manufacturing process and promoter catalyst system that can provide high purity reaction products. Thus, there is a need to address these and other shortcomings associated with existing promoter catalyst systems. These needs and other needs are satisfied by the compositions and methods of the present disclosure.

### SUMMARY

In accordance with the purpose(s) of the invention, as embodied and broadly described herein, this disclosure, in one aspect, relates to catalyst systems, and specifically to promoter ion exchange resin catalyst systems.

In one aspect, the present disclosure provides a process for a chemical condensation reaction, the process comprising contacting at least two chemical reagents with an attached promoter ion exchange resin catalyst system to produce an effluent, and then subjecting the effluent to a solvent crystallization step.

In a second aspect, the present disclosure provides a process comprising contacting at least two chemical reagents with an attached promoter ion exchange resin catalyst system to produce an effluent, and then subjecting the effluent to a solvent crystallization step, wherein the attached promoter ion exchange resin catalyst system comprises cross-linked, sulfonated ion exchange resin having sulfonic groups and a degree of cross-linking of from 1% to 4%.

In a third aspect, the present disclosure provides a process comprising contacting at least two chemical reagents with an attached promoter ion exchange resin catalyst system, wherein the attached promoter ion exchange resin catalyst system comprises a dimethyl thiazolidine promoter.

In a fourth aspect, the present disclosure provides a process comprising contacting at least two chemical reagents with an attached promoter ion exchange resin catalyst system, wherein the attached promoter ion exchange resin catalyst system comprises a promoter that is ionically bound to from about 18% to about 25% of sulfonic acid groups present on the ion exchange resin.

In a fifth aspect, the present disclosure provides a process wherein prior to a solvent crystallization step, a reactor effluent is subjected to at least one of a separate ion exchange resin bed, a water removal step, a phenol recovery step, or a combination thereof.

In a sixth aspect, the present disclosure provides a bisphenol A reaction product having no or substantially no inorganic and/or sulfur impurities.

In another aspect, the present disclosure provides a bisphenol-A monomer having an organic purity of at least about 99.5 wt. % and a sulfur concentration of less than about 5 ppm, wherein when formed into a polycarbonate resin and molded into a 2.5 mm plaque, exhibits a color (YI) of less than about 1.5.

In another aspect, the present disclosure provides a bisphenol-A monomer, having a sulfur concentration of less than about 2 ppm.

In another aspect, the present disclosure provides a bisphenol-A monomer, wherein when formed into a polycarbonate resin and molded into a 2.5 mm plaque, exhibits a color (YI) of less than about 1.3.

In another aspect, the present disclosure provides a bisphenol-A monomer, wherein when formed into a polycarbonate resin and molded into a 2.5 mm plaque, exhibits a color (YI) of less than about 10 after heat aging for 2,000 hours at about 130° C.

In another aspect, the present disclosure provides a bisphenol-A monomer, wherein when formed into a polycarbonate resin and molded into a 2.5 mm plaque, exhibits a color (YI) of less than about 7 after heat aging for 2,000 hours at about 130° C.

In another aspect, the present disclosure provides a bisphenol-A prepared by contacting at least two chemical reagents with an attached promoter ion exchange resin catalyst system to produce an effluent, and then subjecting the effluent to a solvent crystallization step, having a purity level suitable for use in the manufacture of polycarbonate for optical applications and requiring high transmission and low color.

In another aspect, the present disclosure provides a polycarbonate prepared from the bisphenol-A described herein.

### BRIEF DESCRIPTION OF THE FIGURES

The accompanying figures, which are incorporated in and constitute a part of this specification, illustrate several aspects and together with the description serve to explain the principles of the invention.

FIG. 1 illustrates the yellowness index in a plastic 2.5 mm color chip directly after molding as a function of monomer synthesis catalyst & promoter system.

FIG. 2 illustrates the yellowness index in a plastic 2.5 mm color chip after 2,000 hrs of heat aging at 130° C. as a function of monomer synthesis catalyst & promoter system.

FIG. 3 illustrates the yellowness index in a plastic 2.5 mm color chip directly after molding as a function of monomer organic purity and monomer synthesis catalyst & promoter system.

FIG. 4 illustrates the yellowness index in a plastic 2.5 mm color chip after 2,000 hrs of heat aging at 130° C. as a function of monomer organic purity and monomer synthesis catalyst & promoter system.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, example methods and materials are now described.

As used in the specification and the appended claims, the singular forms “a,” “an” and “the” include plural referents unless the context clearly dictates otherwise. Thus, for example, reference to “a ketone” includes mixtures of two or more ketones.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint, and are independently combinable with endpoints of other expressed ranges for the same property. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or can not occur, and that the description includes instances where said event or circumstance occurs and instances where it does not. For example, the phrase “optionally substituted alkyl” means that the alkyl group can or can not be substituted and that the description includes both substituted and unsubstituted alkyl groups.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

References in the specification and concluding claims to parts by weight of a particular element or component in a composition or article denote the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

The term “alkyl group” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is an alkyl group containing from one to six carbon atoms.

The term “alkoxy” as used herein is an alkyl group bound through a single, terminal ether linkage; that is, an “alkoxy” group may be defined as —OR where R is alkyl as defined above. A “lower alkoxy” group is an alkoxy group containing from one to six carbon atoms.

The term “alkenyl group” as used herein is a hydrocarbon group of from 2 to 24 carbon atoms and structural formula containing at least one carbon-carbon double bond. Asymmetric structures such as (AB)C═C(CD) are intended to include both the E and Z isomers. This may be presumed in structural formulae herein wherein an asymmetric alkene is present, or it may be explicitly indicated by the bond symbol C.

The term “alkynyl group” as used herein is a hydrocarbon group of 2 to 24 carbon atoms and a structural formula containing at least one carbon-carbon triple bond.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “aralkyl” as used herein is an aryl group having an alkyl, alkynyl, or alkenyl group as defined above attached to the aromatic group. An example of an aralkyl group is a benzyl group.

The term “hydroxyalkyl group” as used herein is an alkyl, alkenyl, alkynyl, aryl, aralkyl, cycloalkyl, halogenated alkyl, or heterocycloalkyl group described above that has at least one hydrogen atom substituted with a hydroxyl group.

The term “alkoxyalkyl group” is defined as an alkyl, alkenyl, alkynyl, aryl, aralkyl, cycloalkyl, halogenated alkyl, or heterocycloalkyl group described above that has at least one hydrogen atom substituted with an alkoxy group described above.

The term “ester” as used herein is represented by the formula —C(O)OA, where A can be an alkyl, halogenated alkyl, alkenyl, alkynyl, aryl, heteroaryl, cycloalkyl, cycloalkenyl, heterocycloalkyl, or heterocycloalkenyl group described above.

The term “carbonate group” as used herein is represented by the formula —OC(O)OR, where R can be hydrogen, an alkyl, alkenyl, alkynyl, aryl, aralkyl, cycloalkyl, halogenated alkyl, or heterocycloalkyl group described above.

The term “carboxylic acid” as used herein is represented by the formula —C(O)OH.

The term “aldehyde” as used herein is represented by the formula —C(O)H.

The term “keto group” as used herein is represented by the formula —C(O)R, where R is an alkyl, alkenyl, alkynyl, aryl, aralkyl, cycloalkyl, halogenated alkyl, or heterocycloalkyl group described above.

The term “carbonyl group” as used herein is represented by the formula C═O.

The term “ether” as used herein is represented by the formula AOA¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “sulfo-oxo group” as used herein is represented by the formulas —S(O)₂R, —OS(O)₂R, or, —OS(O)₂OR, where R can be hydrogen, an alkyl, alkenyl, alkynyl, aryl, aralkyl, cycloalkyl, halogenated alkyl, or heterocycloalkyl group described above.

As used herein, the term “promoter catalyst system” is intended to refer to a catalyst system comprising a promoter, unless specifically stated to the contrary. A promoter catalyst system can also be referred to as a promoted catalyst system, indicating the presence of a promoter in the catalyst system.

As used herein, unless specifically stated to the contrary, the term “polycarbonate” is intended to refer to compositions having repeating structural carbonate units of formula (1)

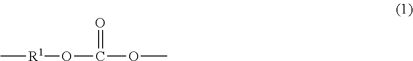

in which at least 60 percent of the total number of R¹ groups contain aromatic moieties and the balance thereof are aliphatic, alicyclic, or aromatic. In an aspect, each R¹ is a C₆₋₃₀ aromatic group, that is, contains at least one aromatic moiety. R¹ can be derived from a dihydroxy compound of the formula HO—R¹—OH, in particular of formula (2)

HO-A¹-Y¹-A²-OH (2)

wherein each of A¹ and A² is a monocyclic divalent aromatic group and Y¹ is a single bond or a bridging group having one or more atoms that separate A¹ from A². In an aspect, one atom separates A¹ from A². Specifically, each R¹ can be derived from a dihydroxy aromatic compound of formula (3)

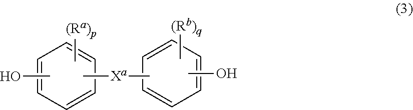

wherein R^{a} and R^{b} are each independently a halogen, C₁₋₁₂ alkoxy, or C₁₋₁₂ alkyl; and p and q are each independently integers of 0 to 4. It will be understood that R^{a} is hydrogen when p is 0, and likewise R^{b} is hydrogen when q is 0. Also in formula (3), X^{a} is a bridging group connecting the two hydroxy-substituted aromatic groups, where the bridging group and the hydroxy substituent of each C₆ arylene group are disposed ortho, meta, or para (specifically para) to each other on the C₆ arylene group. In an aspect, the bridging group X^{a} is single bond, —O—, —S—, —S(O)—, —S(O)₂—, —C(O)—, or a C₁₋₁₈ organic group. The C₁₋₁₈ organic bridging group can be cyclic or acyclic, aromatic or non-aromatic, and can further comprise heteroatoms such as halogens, oxygen, nitrogen, sulfur, silicon, or phosphorous. The C₁₋₁₈ organic group can be disposed such that the C₆ arylene groups connected thereto are each connected to a common alkylidene carbon or to different carbons of the C₁₋₁₈ organic bridging group. In one aspect, p and q is each 1, and R^{a} and R^{b} are each a C₁₋₃ alkyl group, specifically methyl, disposed meta to the hydroxy group on each arylene group.

In another aspect, X^{a} is a substituted or unsubstituted C₃₋₁₈ cycloalkylidene, a C₁₋₂₅ alkylidene of formula —C(R^{c})(R^{d})— wherein R^{e} and R^{d} are each independently hydrogen, C₁₋₁₂ alkyl, C₁₋₁₂ cycloalkyl, C₇₋₁₂ arylalkyl, C₁₋₁₂ heteroalkyl, or cyclic C₇₋₁₂ heteroarylalkyl, or a group of the formula —C(═R^{e})— wherein R^{e} is a divalent C₁₋₁₂ hydrocarbon group. groups of this type include methylene, cyclohexylmethylene, ethylidene, neopentylidene, and isopropylidene, as well as 2-[2.2.1]-bicycloheptylidene, cyclohexylidene, cyclopentylidene, cyclododecylidene, and adamantylidene.

In another aspect, X^{a} is a C₁₋₁₈ alkylene group, a C₃₋₁₈ cycloalkylene group, a fused C₆₋₁₈ cycloalkylene group, or a group of the formula —B¹-G-B²— wherein B¹ and B² are the same or different C₁₋₆ alkylene group and G is a C₃₋₁₂ cycloalkylidene group or a C₆₋₁₆ arylene group. For example, X^{a} can be a substituted C₃₋₁₈ cycloalkylidene of formula (4)

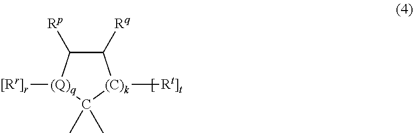

wherein R^{r}, RP, R¹, and R^{t} are each independently hydrogen, halogen, oxygen, or C₁₋₁₂ hydrocarbon groups; Q is a direct bond, a carbon, or a divalent oxygen, sulfur, or —N(Z)— where Z is hydrogen, halogen, hydroxy, C₁₋₁₂ alkyl, C₁₋₁₂ alkoxy, or C₁₋₁₂ acyl; r is 0 to 2, t is 1 or 2, q is 0 or 1, and k is 0 to 3, with the proviso that at least two of R^{r}, R^{p}, R¹, and R^{t} taken together are a fused cycloaliphatic, aromatic, or heteroaromatic ring. It will be understood that where the fused ring is aromatic, the ring as shown in formula (4) will have an unsaturated carbon-carbon linkage where the ring is fused. When k is one and i is 0, the ring as shown in formula (4) contains 4 carbon atoms, when k is 2, the ring as shown in formula (4) contains 5 carbon atoms, and when k is 3, the ring contains 6 carbon atoms. In an aspect, two adjacent groups (e.g., R^{q} and R^{t} taken together) form an aromatic group, and in another aspect, R^{q} and R^{t} taken together form one aromatic group and R^{r} and R^{p} taken together form a second aromatic group. When R^{q} and R^{t} taken together form an aromatic group, R^{p} can be a double-bonded oxygen atom, i.e., a ketone.

In one aspect, bisphenols (4) can be used in the manufacture of polycarbonates containing phthalimidine carbonate units of formula (4a)

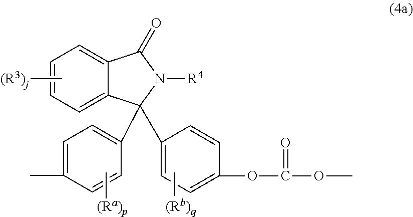

wherein R^{a}, R^{b}, p, and q are as in formula (4), R³ is each independently a C₁₋₆ alkyl group, j is 0 to 4, and R₄ is a C₁₋₆ alkyl, phenyl, or phenyl substituted with up to five C₁₋₆ alkyl groups. In particular, the phthalimidine carbonate units are of formula (4b)

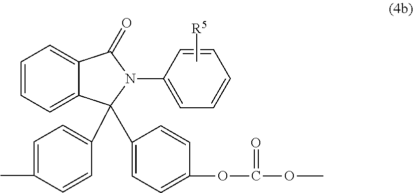

wherein R⁵ is hydrogen or a C₁₋₆ alkyl. In an aspect, R⁵ is hydrogen. Carbonate units (4a) wherein R⁵ is hydrogen can be derived from 2-phenyl-3,3′-bis(4-hydroxy phenyl)phthalimidine (also known as N-phenyl phenolphthalein bisphenol, or “PPPBP”) (also known as 3,3-bis(4-hydroxyphenyl)-2-phenylisoindolin-1-one).

Other bisphenol carbonate repeating units of this type are the isatin carbonate units of formula (4c) and (4d)

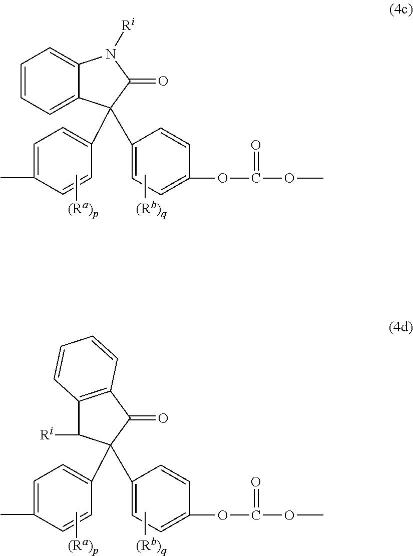

wherein R^{a} and R^{b} are each independently C₁₋₁₂ alkyl, p and q are each independently 0 to 4, and R′ is C₁₋₁₂ alkyl, phenyl, optionally substituted with 15 to C₁₋₁₀ alkyl, or benzyl optionally substituted with 1 to 5 C₁₋₁₀ alkyl. In an aspect, R^{a} and R^{b} are each methyl, p and q are each independently 0 or 1, and R′ is C₁₋₄ alkyl or phenyl.

Examples of bisphenol carbonate units derived from bisphenols (4) wherein X^{b} is a substituted or unsubstituted C₃₋₁₈ cycloalkylidene include the cyclohexylidene-bridged, alkyl-substituted bisphenol of formula (4e)

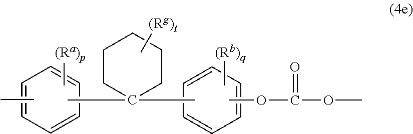

wherein R^{a} and R^{b} are each independently C₁₋₁₂ alkyl, R^{g} is C₁₋₁₂ alkyl, p and q are each independently 0 to 4, and t is 0 to 10. In a specific aspect, at least one of each of R^{a} and R^{b} are disposed meta to the cyclohexylidene bridging group. In another aspect, R^{a} and R^{b} are each independently C₁₋₄ alkyl, R^{g} is C₁₋₄ alkyl, p and q are each 0 or 1, and t is 0 to 5. In another aspect, R^{a}, R^{b}, and R^{g} are each methyl, r and s are each 0 or 1, and t is 0 or 3, specifically 0. For example,

Examples of other bisphenol carbonate units derived from bisphenol (4) wherein X^{b} is a substituted or unsubstituted C₃₋₁₈ cycloalkylidene include adamantyl units (4f) and units (4g)

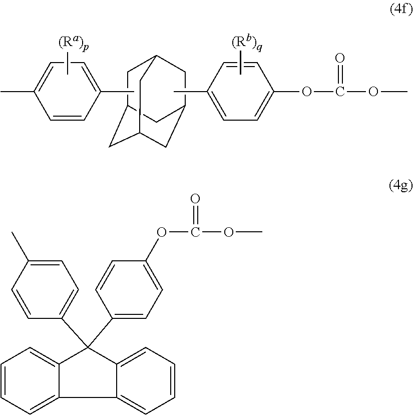

wherein R^{a} and R^{b} are each independently C₁₋₁₂ alkyl, and p and q are each independently 1 to 4. In a specific aspect, at least one of each of R^{a} and R^{b} are disposed meta to the cycloalkylidene bridging group. In an aspect, R^{a} and R^{b} are each independently C₁₋₃ alkyl, and p and q are each 0 or 1. In another specific aspect, R^{a}, R^{b} are each methyl, p and q are each 0 or 1. Carbonates containing units (4a) to (4g) are useful for making polycarbonates with high glass transition temperatures (Tg) and high heat distortion temperatures.

Other useful aromatic dihydroxy compounds of the formula HO—R¹—OH include compounds of formula (5)

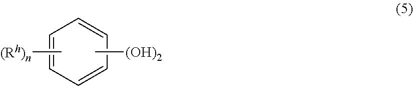

wherein each R^{h} is independently a halogen atom, a C₁₋₁₀ hydrocarbyl such as a C₁₋₁₀ alkyl group, a halogen-substituted C₁₋₁₀ alkyl group, a C₆₋₁₀ aryl group, or a halogen-substituted C₆₋₁₀ aryl group, and n is 0 to 4. The halogen is usually bromine.

Some illustrative examples of specific aromatic dihydroxy compounds include the following: 4,4′-dihydroxybiphenyl, 1,6-dihydroxynaphthalene, 2,6-dihydroxynaphthalene, bis(4-hydroxyphenyl)methane, bis(4-hydroxyphenyl)diphenylmethane, bis(4-hydroxyphenyl)-1-naphthylmethane, 1,2-bis(4-hydroxyphenyl)ethane, 1,1-bis(4-hydroxyphenyl)-1-phenylethane, 2-(4-hydroxyphenyl)-2-(3-hydroxyphenyl)propane, bis(4-hydroxyphenyl)phenylmethane, 2,2-bis(4-hydroxy-3-bromophenyl)propane, 1,1-bis(hydroxyphenyl)cyclopentane, 1,1-bis(4-hydroxyphenyl)cyclohexane, 1,1-bis(4-hydroxyphenyl)isobutene, 1,1-bis(4-hydroxyphenyl)cyclododecane, trans-2,3-bis(4-hydroxyphenyl)-2-butene, 2,2-bis(4-hydroxyphenyl)adamantane, alpha,alpha′-bis(4-hydroxyphenyl)toluene, bis(4-hydroxyphenyl)acetonitrile, 2,2-bis(3-methyl-4-hydroxyphenyl)propane, 2,2-bis(3-ethyl-4-hydroxyphenyl)propane, 2,2-bis(3-n-propyl-4-hydroxyphenyl)propane, 2,2-bis(3-isopropyl-4-hydroxyphenyl)propane, 2,2-bis(3-sec-butyl-4-hydroxyphenyl)propane, 2,2-bis(3-t-butyl-4-hydroxyphenyl)propane, 2,2-bis(3-cyclohexyl-4-hydroxyphenyl)propane, 2,2-bis(3-allyl-4-hydroxyphenyl)propane, 2,2-bis(3-methoxy-4-hydroxyphenyl)propane, 2,2-bis(4-hydroxyphenyl)hexafluoropropane, 1,1-dichloro-2,2-bis(4-hydroxyphenyl)ethylene, 1,1-dibromo-2,2-bis(4-hydroxyphenyl)ethylene, 1,1-dichloro-2,2-bis(5-phenoxy-4-hydroxyphenyl)ethylene, 4,4′-dihydroxybenzophenone, 3,3-bis(4-hydroxyphenyl)-2-butanone, 1,6-bis(4-hydroxyphenyl)-1,6-hexanedione, ethylene glycol bis(4-hydroxyphenyl)ether, bis(4-hydroxyphenyl)ether, bis(4-hydroxyphenyl)sulfide, bis(4-hydroxyphenyl)sulfoxide, bis(4-hydroxyphenyl)sulfone, 9,9-bis(4-hydroxyphenyl)fluorine, 2,7-dihydroxypyrene, 6,6′-dihydroxy-3,3,3′,3′-tetramethylspiro(bis)indane (“spirobiindane bisphenol”), 3,3-bis(4-hydroxyphenyl)phthalimide, 2,6-dihydroxydibenzo-p-dioxin, 2,6-dihydroxythianthrene, 2,7-dihydroxyphenoxathin, 2,7-dihydroxy-9,10-dimethylphenazine, 3,6-dihydroxydibenzofuran, 3,6-dihydroxydibenzothiophene, and 2,7-dihydroxycarbazole, resorcinol, substituted resorcinol compounds such as 5-methyl resorcinol, 5-ethyl resorcinol, 5-propyl resorcinol, 5-butyl resorcinol, 5-t-butyl resorcinol, 5-phenyl resorcinol, 5-cumyl resorcinol, 2,4,5,6-tetrafluoro resorcinol, 2,4,5,6-tetrabromo resorcinol, or the like; catechol; hydroquinone; substituted hydroquinones such as 2-methyl hydroquinone, 2-ethyl hydroquinone, 2-propyl hydroquinone, 2-butyl hydroquinone, 2-t-butyl hydroquinone, 2-phenyl hydroquinone, 2-cumyl hydroquinone, 2,3,5,6-tetramethyl hydroquinone, 2,3,5,6-tetra-t-butyl hydroquinone, 2,3,5,6-tetrafluoro hydroquinone, 2,3,5,6-tetrabromo hydroquinone, or the like, or combinations comprising at least one of the foregoing dihydroxy compounds.

Specific examples of bisphenol compounds of formula (3) include 1,1-bis(4-hydroxyphenyl)methane, 1,1-bis(4-hydroxyphenyl)ethane, 2,2-bis(4-hydroxyphenyl)propane (hereinafter “bisphenol A” or “BPA”), 2,2-bis(4-hydroxyphenyl)butane, 2,2-bis(4-hydroxyphenyl)octane, 1,1-bis(4-hydroxyphenyl)propane, 1,1-bis(4-hydroxyphenyl)n-butane, 2,2-bis(4-hydroxy-2-methylphenyl)propane, 1,1-bis(4-hydroxy-t-butylphenyl)propane, 3,3-bis(4-hydroxyphenyl)phthalimidine, 2-phenyl-3,3-bis(4-hydroxyphenyl)phthalimidine (PPPBP), and 1,1-bis(4-hydroxy-3-methylphenyl)cyclohexane (DMBPC). Combinations comprising at least one of the foregoing dihydroxy compounds can also be used. In one specific aspect, the polycarbonate is a linear homopolymer derived from bisphenol A, in which each of A¹ and A² is p-phenylene and Y¹ is isopropylidene in formula (3).

Further to the description above, the term “polycarbonates” is intended to refer to homopolycarbonates (wherein each R¹ in the polymer is the same), copolymers comprising different R¹ moieties in the carbonate (“copolycarbonates”), copolymers comprising carbonate units and other types of polymer units, such as ester units, and combinations comprising at least one of homopolycarbonates and/or copolycarbonates.

A specific type of copolymer is a polyester carbonate, also known as a polyester-polycarbonate. Such copolymers further contain, in addition to recurring carbonate chain units of formula (1), repeating units of formula (6)

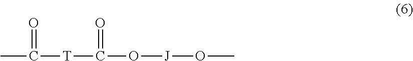

wherein J is a divalent group derived from a dihydroxy compound, and can be, for example, a C₂₋₁₀ alkylene, a C₆₋₂₀ cycloalkylene a C₆₋₂₀ arylene, or a polyoxyalkylene group in which the alkylene groups contain 2 to 6 carbon atoms, specifically 2, 3, or 4 carbon atoms; and T is a divalent group derived from a dicarboxylic acid, and can be, for example, a C₂₋₁₀ alkylene, a C₆₋₂₀ cycloalkylene, or a C₆₋₂₀ arylene. Copolyesters containing a combination of different T and/or J groups can be used. The polyesters can be branched or linear.

In an aspect, J is a C₂₋₃₀ alkylene group having a straight chain, branched chain, or cyclic (including polycyclic) structure. In another aspect, J is derived from an aromatic dihydroxy compound of formula (3) above. In another aspect, J is derived from an aromatic dihydroxy compound of formula (4) above. In another aspect, J is derived from an aromatic dihydroxy compound of formula (5) above.

Aromatic dicarboxylic acids that can be used to prepare the polyester units include isophthalic or terephthalic acid, 1,2-di(p-carboxyphenyl)ethane, 4,4′-dicarboxydiphenyl ether, 4,4′-bisbenzoic acid, or a combination comprising at least one of the foregoing acids. Acids containing fused rings can also be present, such as in 1,4-, 1,5-, or 2,6-naphthalenedicarboxylic acids. Specific dicarboxylic acids include terephthalic acid, isophthalic acid, naphthalene dicarboxylic acid, cyclohexane dicarboxylic acid, or a combination comprising at least one of the foregoing acids. A specific dicarboxylic acid comprises a combination of isophthalic acid and terephthalic acid wherein the weight ratio of isophthalic acid to terephthalic acid is 91:9 to 2:98. In another specific aspect, J is a C₂₋₆ alkylene group and T is p-phenylene, m-phenylene, naphthalene, a divalent cycloaliphatic group, or a combination thereof. This class of polyester includes the poly(alkylene terephthalates).

The molar ratio of ester units to carbonate units in the copolymers can vary broadly, for example 1:99 to 99:1, specifically 10:90 to 90:10, more specifically 25:75 to 75:25, depending on the desired properties of the final composition.

In a specific aspect, the polyester unit of a polyester-polycarbonate is derived from the reaction of a combination of isophthalic and terephthalic diacids (or derivatives thereof) with resorcinol. In another specific aspect, the polyester unit of a polyester-polycarbonate is derived from the reaction of a combination of isophthalic acid and terephthalic acid with bisphenol A. In a specific aspect, the polycarbonate units are derived from bisphenol A. In another specific aspect, the polycarbonate units are derived from resorcinol and bisphenol A in a molar ratio of resorcinol carbonate units to bisphenol A carbonate units of 1:99 to 99:1.

Polycarbonates can be manufactured by processes such as interfacial polymerization and melt polymerization. Branched polycarbonate blocks can be prepared by adding a branching agent during polymerization. These branching agents include polyfunctional organic compounds containing at least three functional groups selected from hydroxyl, carboxyl, carboxylic anhydride, haloformyl, and mixtures of the foregoing functional groups. Specific examples include trimellitic acid, trimellitic anhydride, trimellitic trichloride, tris-p-hydroxy phenyl ethane, isatin-bis-phenol, tris-phenol TC (1,3,5-tris((p-hydroxyphenyl)isopropyl)benzene), tris-phenol PA (4(4(1,1-bis(p-hydroxyphenyl)-ethyl) alpha,alpha-dimethyl benzyl)phenol), 4-chloroformyl phthalic anhydride, trimesic acid, and benzophenone tetracarboxylic acid. The branching agents can be added at a level of 0.05 to 2.0 wt %. Mixtures comprising linear polycarbonates and branched polycarbonates can be used.

A chain stopper (also referred to as a capping agent) can be included during polymerization. The chain stopper limits molecular weight growth rate, and so controls molecular weight in the polycarbonate. chain stoppers include certain mono-phenolic compounds, mono-carboxylic acid chlorides, and/or mono-chloroformates. Mono-phenolic chain stoppers are exemplified by monocyclic phenols such as phenol and C₁-C₂₂ alkyl-substituted phenols such as p-cumyl-phenol, resorcinol monobenzoate, and p- and tertiary-butyl phenol; and monoethers of diphenols, such as p-methoxyphenol. Alkyl-substituted phenols with branched chain alkyl substituents having 8 to 9 carbon atom can be specifically mentioned. Certain mono-phenolic UV absorbers can also be used as a capping agent, for example 4-substituted-2-hydroxybenzophenones and their derivatives, aryl salicylates, monoesters of diphenols such as resorcinol monobenzoate, 2-(2-hydroxyaryl)-benzotriazoles and their derivatives, 2-(2-hydroxyaryl)-1,3,5-triazines and their derivatives, and the like.

Mono-carboxylic acid chlorides can also be used as chain stoppers. These include monocyclic, mono-carboxylic acid chlorides such as benzoyl chloride, C₁-C₂₂ alkyl-substituted benzoyl chloride, toluoyl chloride, halogen-substituted benzoyl chloride, bromobenzoyl chloride, cinnamoyl chloride, 4-nadimidobenzoyl chloride, and combinations thereof; polycyclic, mono-carboxylic acid chlorides such as trimellitic anhydride chloride, and naphthoyl chloride; and combinations of monocyclic and polycyclic mono-carboxylic acid chlorides. Chlorides of aliphatic monocarboxylic acids with less than or equal to 22 carbon atoms are useful. Functionalized chlorides of aliphatic monocarboxylic acids, such as acryloyl chloride and methacryoyl chloride, are also useful. Also useful are mono-chloroformates including monocyclic, mono-chloroformates, such as phenyl chloroformate, alkyl-substituted phenyl chloroformate, p-cumyl phenyl chloroformate, toluene chloroformate, and combinations thereof.

Alternatively, melt processes can be used to make the polycarbonates. The polyester-polycarbonates can also be prepared by interfacial polymerization. Rather than utilizing the dicarboxylic acid or diol per se, the reactive derivatives of the acid or diol, such as the corresponding acid halides, in particular the acid dichlorides and the acid dibromides can be used. Thus, for example instead of using isophthalic acid, terephthalic acid, or a combination comprising at least one of the foregoing acids, isophthaloyl dichloride, terephthaloyl dichloride, or a combination comprising at least one of the foregoing dichlorides can be used.

In addition to the polycarbonates described above, combinations of the polycarbonate with other thermoplastic polymers, for example combinations of homopolycarbonates and/or polycarbonate copolymers with polyesters, can be used. Useful polyesters can include, for example, polyesters having repeating units of formula (6), which include poly(alkylene dicarboxylates), liquid crystalline polyesters, and polyester copolymers. The polyesters described herein are generally completely miscible with the polycarbonates when blended.

The polyesters can be obtained by interfacial polymerization or melt-process condensation as described above, by solution phase condensation, or by transesterification polymerization wherein, for example, a dialkyl ester such as dimethyl terephthalate can be transesterified with ethylene glycol using acid catalysis, to generate poly(ethylene terephthalate). A branched polyester, in which a branching agent, for example, a glycol having three or more hydroxyl groups or a trifunctional or multifunctional carboxylic acid has been incorporated, can be used. Furthermore, it can be desirable to have various concentrations of acid and hydroxyl end groups on the polyester, depending on the ultimate end use of the composition.

Useful polyesters can include aromatic polyesters, poly(alkylene esters) including poly(alkylene arylates), and poly(cycloalkylene diesters). Aromatic polyesters can have a polyester structure according to formula (6), wherein J and T are each aromatic groups as described hereinabove. In an aspect, useful aromatic polyesters can include, for example, poly(isophthalate-terephthalate-resorcinol) esters, poly(isophthalate-terephthalate-bisphenol A) esters, poly[(isophthalate-terephthalate-resorcinol) ester-co-(isophthalate-terephthalate-bisphenol A)]ester, or a combination comprising at least one of these. Also contemplated are aromatic polyesters with a minor amount, e.g., 0.5 to 10 weight percent, based on the total weight of the polyester, of units derived from an aliphatic diacid and/or an aliphatic polyol to make copolyesters. Poly(alkylene arylates) can have a polyester structure according to formula (6), wherein T comprises groups derived from aromatic dicarboxylates, cycloaliphatic dicarboxylic acids, or derivatives thereof. Examples of specifically useful T groups include 1,2-, 1,3-, and 1,4-phenylene; 1,4- and 1,5-naphthylenes; cis- or trans-1,4-cyclohexylene; and the like. Specifically, where T is 1,4-phenylene, the poly(alkylene arylate) is a poly(alkylene terephthalate). In addition, for poly(alkylene arylate), specifically useful alkylene groups J include, for example, ethylene, 1,4-butylene, and bis-(alkylene-disubstituted cyclohexane) including cis- and/or trans-1,4-(cyclohexylene)dimethylene. Examples of poly(alkylene terephthalates) include poly(ethylene terephthalate) (PET), poly(1,4-butylene terephthalate) (PBT), and poly(propylene terephthalate) (PPT). Also useful are poly(alkylene naphthoates), such as poly(ethylene naphthanoate) (PEN), and poly(butylene naphthanoate) (PBN). A specifically useful poly(cycloalkylene diester) is poly(cyclohexanedimethylene terephthalate) (PCT). Combinations comprising at least one of the foregoing polyesters can also be used.

Copolymers comprising alkylene terephthalate repeating ester units with other ester groups can also be useful. Specifically useful ester units can include different alkylene terephthalate units, which can be present in the polymer chain as individual units, or as blocks of poly(alkylene terephthalates). copolymers of this type include poly(cyclohexanedimethylene terephthalate)-co-poly(ethylene terephthalate), abbreviated as PETG where the polymer comprises greater than or equal to 50 mol % of poly(ethylene terephthalate), and abbreviated as PCTG where the polymer comprises greater than 50 mol % of poly(1,4-cyclohexanedimethylene terephthalate).

Poly(cycloalkylene diester)s can also include poly(alkylene cyclohexanedicarboxylate)s. Of these, a specific example is poly(1,4-cyclohexane-dimethanol-1,4-cyclohexanedicarboxylate) (PCCD), having recurring units of formula (7)

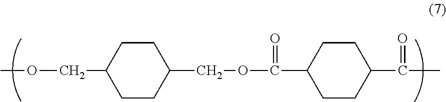

wherein, as described using formula (6), J is a 1,4-cyclohexanedimethylene group derived from 1,4-cyclohexanedimethanol, and T is a cyclohexane ring derived from cyclohexanedicarboxylate or a chemical equivalent thereof, and can comprise the cis-isomer, the trans-isomer, or a combination comprising at least one of the foregoing isomers.

The polycarbonate and polyester can be used in a weight ratio of 1:99 to 99:1, specifically 10:90 to 90:10, and more specifically 30:70 to 70:30, depending on the function and properties desired.

It is desirable for such a polyester and polycarbonate blend to have an MVR of 5 to 150 cc/10 min, specifically 7 to 125 cc/10 min, more specifically 9 to 110 cc/10 min, and still more specifically 10 to 100 cc/10 min, measured at 300° C. and a load of 1.2 kilograms according to ASTM D1238-04.

In another aspect, a polycarbonate can comprise a polysiloxane-polycarbonate copolymer, also referred to as a polysiloxane-polycarbonate. The polydiorganosiloxane (also referred to herein as “polysiloxane”) blocks of the copolymer comprise repeating diorganosiloxane units as in formula (8)

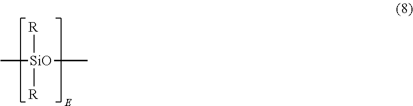

wherein each R is independently a C₁₋₁₃ monovalent organic group. For example, R can be a C₁-C₁₃ alkyl, C₁-C₁₃ alkoxy, C₂-C₁₃ alkenyl group, C₂-C₁₃ alkenyloxy, C₃-C₆ cycloalkyl, C₃-C₆ cycloalkoxy, C₆-C₁₄ aryl, C₆-C₁₀ aryloxy, C₇-C₁₃ arylalkyl, C₇-C₁₃ aralkoxy, C₇-C₁₃ alkylaryl, or C₇-C₁₃ alkylaryloxy. The foregoing groups can be fully or partially halogenated with fluorine, chlorine, bromine, or iodine, or a combination thereof. In an aspect, where a transparent polysiloxane-polycarbonate is desired, R is unsubstituted by halogen. Combinations of the foregoing R groups can be used in the same copolymer.

The value of E in formula (8) can vary widely depending on the type and relative amount of each component in the thermoplastic composition, the desired properties of the composition, and like considerations. Generally, E has an average value of 2 to 1,000, specifically 2 to 500, or 2 to 200, more specifically 5 to 100. In an aspect, E has an average value of 10 to 75, and in still another aspect, E has an average value of 40 to 60. Where E is of a lower value, e.g., less than 40, it can be desirable to use a relatively larger amount of the polycarbonate-polysiloxane copolymer. Conversely, where E is of a higher value, e.g., greater than 40, a relatively lower amount of the polycarbonate-polysiloxane copolymer can be used.

A combination of a first and a second (or more) polycarbonate-polysiloxane copolymers can be used, wherein the average value of E of the first copolymer is less than the average value of E of the second copolymer.

In an aspect, the polydiorganosiloxane blocks are of formula (9)

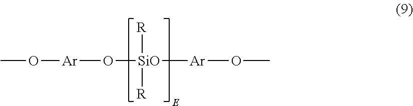

wherein E is as defined above; each R can be the same or different, and is as defined above; and Ar can be the same or different, and is a substituted or unsubstituted C₆-C₃₀ arylene group, wherein the bonds are directly connected to an aromatic moiety. Ar groups in formula (9) can be derived from a C₆-C₃₀ dihydroxyarylene compound, for example a dihydroxyarylene compound of formula (3) or (5) above. dihydroxyarylene compounds are 1,1-bis(4-hydroxyphenyl)methane, 1,1-bis(4-hydroxyphenyl)ethane, 2,2-bis(4-hydroxyphenyl)propane, 2,2-bis(4-hydroxyphenyl)butane, 2,2-bis(4-hydroxyphenyl)octane, 1,1-bis(4-hydroxyphenyl)propane, 1,1-bis(4-hydroxyphenyl)n-butane, 2,2-bis(4-hydroxy-1-methylphenyl)propane, 1,1-bis(4-hydroxyphenyl)cyclohexane, bis(4-hydroxyphenyl sulfide), and 1,1-bis(4-hydroxy-t-butylphenyl)propane. Combinations comprising at least one of the foregoing dihydroxy compounds can also be used.

In another aspect, polydiorganosiloxane blocks are of formula (10)

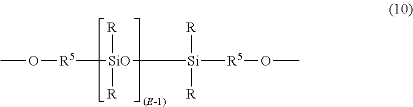

wherein R and E are as described above, and each R⁵ is independently a divalent C₁-C₃₀ organic group, and wherein the polymerized polysiloxane unit is the reaction residue of its corresponding dihydroxy compound. In a specific aspect, the polydiorganosiloxane blocks are of formula (11):

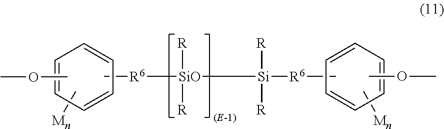

wherein R and E are as defined above. R⁶ in formula (11) is a divalent C₂-C₈ aliphatic group. Each M in formula (11) can be the same or different, and can be a halogen, cyano, nitro, C₁-C₈ alkylthio, C₁-C₈ alkyl, C₁-C₈ alkoxy, C₂-C₈ alkenyl, C₂-C₈ alkenyloxy group, C₃-C₈ cycloalkyl, C₃-C₈ cycloalkoxy, C₆-C₁₀ aryl, C₆-C₁₀ aryloxy, C₇-C₁₂ aralkyl, C₇-C₁₂ aralkoxy, C₇-C₁₂ alkylaryl, or C₇-C₁₂ alkylaryloxy, wherein each n is independently 0, 1, 2, 3, or 4.

In an aspect, M is bromo or chloro, an alkyl group such as methyl, ethyl, or propyl, an alkoxy group such as methoxy, ethoxy, or propoxy, or an aryl group such as phenyl, chlorophenyl, or tolyl; R² is a dimethylene, trimethylene or tetramethylene group; and R is a C₁₋₈ alkyl, haloalkyl such as trifluoropropyl, cyanoalkyl, or aryl such as phenyl, chlorophenyl or tolyl. In another aspect, R is methyl, or a combination of methyl and trifluoropropyl, or a combination of methyl and phenyl. In still another aspect, M is methoxy, n is one, R² is a divalent C₁-C₃ aliphatic group, and R is methyl.

Blocks of formula (11) can be derived from the corresponding dihydroxy polydiorganosiloxane (12)

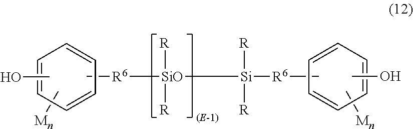

wherein R, E, M, R⁶, and n are as described above. Such dihydroxy polysiloxanes can be made by effecting a platinum-catalyzed addition between a siloxane hydride of formula (13)

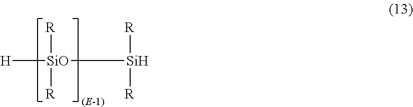

wherein R and E are as previously defined, and an aliphatically unsaturated monohydric phenol. aliphatically unsaturated monohydric phenols include eugenol, 2-alkylphenol, 4-allyl-2-methylphenol, 4-allyl-2-phenylphenol, 4-allyl-2-bromophenol, 4-allyl-2-t-butoxyphenol, 4-phenyl-2-phenylphenol, 2-methyl-4-propylphenol, 2-allyl-4,6-dimethylphenol, 2-allyl-4-bromo-6-methylphenol, 2-allyl-6-methoxy-4-methylphenol and 2-allyl-4,6-dimethylphenol. Combinations comprising at least one of the foregoing can also be used.

The polyorganosiloxane-polycarbonate can comprise 50 to 99 weight percent of carbonate units and 1 to 50 weight percent siloxane units. Within this range, the polyorganosiloxane-polycarbonate copolymer can comprise 70 to 98 weight percent, more specifically 75 to 97 weight percent of carbonate units and 2 to 30 weight percent, more specifically 3 to 25 weight percent siloxane units.

Polyorganosiloxane-polycarbonates can have a weight average molecular weight of 2,000 to 100,000 Daltons, specifically 5,000 to 50,000 Daltons as measured by gel permeation chromatography using a crosslinked styrene-divinyl benzene column, at a sample concentration of 1 milligram per milliliter, and as calibrated with polycarbonate standards.

The polyorganosiloxane-polycarbonate can have a melt volume flow rate, measured at 300° C./1.2 kg, of 1 to 50 cubic centimeters per 10 minutes (cc/10 min), specifically 2 to 30 cc/10 min Mixtures of polyorganosiloxane-polycarbonates of different flow properties can be used to achieve the overall desired flow property.

In another aspect, a polycarbonate material can comprise a flame retardant. In another aspect, a BPA polycarbonate material can comprise a second polycarbonate derived from bisphenol-A, wherein the second polycarbonate is different than the BPA polycarbonate. In another aspect, a BPA polycarbonate material can comprise a second polycarbonate derived from bisphenol-A, wherein the second polycarbonate is selected from at least one of the following: a homopolycarbonate derived from a bisphenol; a copolycarbonate derived from more than on bisphenol; and a copolymer derived from one or more bisphenols and comprising one or more aliphatic ester units or aromatic ester units or siloxane units. In still another aspect, a BPA polycarbonate can comprise one or more additives selected from at least one of the following: UV stabilizing additives, thermal stabilizing additives, mold release agents, colorants, organic fillers, inorganic fillers, and gamma-stabilizing agents.

Each of the materials disclosed herein are either commercially available and/or the methods for the production thereof are known to those of skill in the art.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As briefly described above, the present disclosure provides a manufacturing process and a promoter catalyst system that can be useful in condensation reactions, such as, for example, the synthesis of bisphenol-A. Conventional ion exchange resin based BPA manufacturing processes utilize sulfur containing bulk promoters, such as 3-mercaptopropionic acid (3-MPA), that can degrade and generate undesirable sulfur compounds in the final product. These compounds can limit or prevent the use of BPA in demanding applications, such as food contact grade polycarbonate.

In one aspect, the present disclosure provides a manufacturing process that can produce high purity BPA, with no or substantially no inorganic, sulfur, or thermally degraded components. In one aspect, the present disclosure provides a manufacturing process that can produce high purity BPA having low or no sulfur present. In another aspect, the present disclosure provides a manufacturing process that does not utilize a bulk promoter, such as, for example, 3-MPA. In yet another aspect, the present disclosure provides a manufacturing process and catalyst system that can provide high purity BPA, suitable for use in food contact polycarbonate applications, healthcare applications, optical applications, or a combination thereof. In still another aspect, the present disclosure provides a manufacturing process that comprises an attached promoter catalyst in combination with solvent crystallization.

Promoter systems can also be attached, wherein the promoter is attached to the catalyst system, such as the ion exchange resin. An exemplary attached promoter system utilizes a pyridyl ethylmercapton (PEM) promoter.

In one aspect, the methods described here can be useful for the preparation of BPA. It should also be noted that reactants for bisphenol condensation reactions can comprise phenols, ketones and/or aldehydes, or mixtures thereof. In one aspect, any specific recitation of a ketone, such as acetone, or an aldehyde, is intended to include aspects where only the recited species is used, aspects wherein the other species (e.g., aldehyde for ketone) is used, and aspects wherein a combination of species is used. In other aspects, the methods described herein can be useful for the preparation of other chemical species from, for example, condensation reactions.

In one aspect, phenol reactants can comprise an aromatic hydroxy compound having at least one unsubstituted position, and optionally one or more inert substituents such as hydrocarbyl or halogen at one or more ring positions. In one aspect, an inert substituent is a substituent which does not interfere undesirably with the condensation of the phenol and ketone or aldehyde and which is not, itself, catalytic. In another aspect, phenol reactants are unsubstituted in the position para to the hydroxyl group. As recited here, hydrocarbyl functionalities comprise carbon and hydrogen atoms, such as, for example, alkylene, alkyl, cycloaliphatic, aryl, arylene, alkylarylene, arylalkylene, alkylcycloaliphatic and alkylenecycloaliphatic are hydrocarbyl functions, that is, functions containing carbon and hydrogen atoms.

In one aspect, an alkyl group, if present in a phenol species, comprises from 1 to about 20 carbon atoms, or from 1 to about 5 carbon atoms, or from 1 to about 3 carbon atoms, such as, for example, various methyl, ethyl, propyl, butyl and pentyl isomers. In one aspect, alkyl, aryl, alkaryl and aralkyl substituents are suitable hydrocarbyl substituents on the phenol reactant.

In one aspect, other inert phenol substituents can include, but are not limited to alkoxy, aryloxy or alkaryloxy, wherein alkoxy includes methoxy, ethoxy, propyloxy, butoxy, pentoxy, hexoxy, heptoxy, octyloxy, nonyloxy, decyloxy and polyoxyethylene, as well as higher homologues; aryloxy, phenoxy, biphenoxy, naphthyloxy, etc. and alkaryloxy includes alkyl, alkenyl and alkylnyl-substituted phenolics. Additional inert phenol substituents can include halo, such as bromo, chloro or iodo.

While not intending to be limiting, exemplary phenols can comprise, phenol, 2-cresol, 3-cresol, 4-cresol, 2-chlorophenol, 3-chlorophenol, 4-chlorophenol, 2-tert-butylphenol, 2,4-dimethylphenol, 2-ethyl-6-methylphenol, 2-bromophenol, 2-fluorophenol, 2-phenoxyphenol, 3-methoxyphenol, 2,3,6-trimethylphenol, 2,3,5,6-tetramethylphenol, 2,6-xylenol, 2,6-dichlorophenol, 3,5-diethylphenol, 2-benzylphenol, 2,6-di-tertbutylphenol, 2-phenylphenol, 1-naphthol, 2-naphthol, and/or combinations thereof. In another aspect, phenol reactants can comprise phenol, 2- or 3-cresol, 2,6-dimethylphenol, resorcinol, naphthols, and/or combinations or mixtures thereof. In one aspect, a phenol is unsubstituted.

In one aspect, the phenol starting materials can be commercial grade or better. As readily understood by one of ordinary skill in the art commercial grade reagents may contain measurable levels of typical impurities such as acetone, alpha-methylstyrene, acetophenone, alkyl benzenes, cumene, cresols, water, hydroxyacetone, methyl benzofuran, methyl cyclopentenone, and mesityl oxide, among others.

In one aspect, ketones, if used, can comprise any ketone having a single carbonyl (C═O) group or several carbonyl groups, and which are reactive under the conditions used. In another aspect, ketones can be substituted with substituents that are inert under the conditions used, such as, for example those inert substituents recited above with respect to phenols.

In one aspect, a ketone can comprise aliphatic, aromatic, alicyclic or mixed aromatic-aliphatic ketones, diketones or polyketones, of which acetone, methyl ethyl ketone, diethyl ketone, benzyl, acetyl acetone, methyl isopropyl ketone, methyl isobutyl ketone, acetophenone, ethyl phenyl ketone, cyclohexanone, cyclopentanone, benzophenone, fluorenone, indanone, 3,3,5-trimethylcyclohexanone, anthraquinone, 4-hydroxyacetophenone, acenaphthenequinone, quinone, benzoylacetone and diacetyl are representative examples. In another aspect, a ketone having halo, nitrile or nitro substituents can also be used, for example, 1,3-dichloroacetone or hexafluoroacetone.

Exemplary aliphatic ketones can comprise acetone, ethyl methyl ketone, isobutyl methyl ketone, 1,3-dichloroacetone, hexafluoroacetone, or combinations thereof. In one aspect, the ketone is acetone, which can condense with phenol to produce 2,2-bis-(4-hydroxyphenyl)-propane, commonly known as bisphenol A. In another aspect, a ketone comprises hexafluoroacetone, which can react with two moles of phenol to produce 2,2-bis-(4-hydroxyphenyl)-hexafluoropropane (bisphenol AF). In another aspect, a ketone can comprise a ketone having at least one hydrocarbyl group containing an aryl group, for example, a phenyl, tolyl, naphthyl, xylyl or 4-hydroxyphenyl group.

Other exemplary ketones can include 9-fluorenone, cyclohexanone, 3,3,5-trimethylcyclohexanone, indanone, indenone, anthraquinone, or combinations thereof. Still other exemplary ketones can include benzophenone, acetophenone, 4-hydroxyacetophenone, 4,4′-dihydroxybenzophenone, or combinations thereof.

In one aspect, a ketone reactant can be commercial grade or better. As readily understood by one of ordinary skill in the art commercial grade reagents may contain measurable levels of typical impurities such as aldehydes, acetophenone, benzene, cumene, diacetone alcohol, water, mesityl oxide, and methanol, among others. In one aspect, a ketone, such as, for example, acetone, has less than about 250 ppm of methanol. In another aspect, the inventive catalyst systems of the present invention can tolerate higher concentrations of impurities, such that a ketone can comprise more than 250 ppm of methanol.

In other aspects, the various methods and catalyst systems described herein can be used for the condensation of phenols with aldehydes, for example, with formaldehyde, acetaldehyde, propionaldehyde, butyraldehyde or higher homologues of the formula RCHO, wherein R is alkyl of, for example, 1 to 20 carbon atoms. In one aspect, the condensation of two moles of phenol with one mole of formaldehyde produces bis-(4-hydroxyphenyl)methane, also known as Bisphenol F. It should also be understood that dialdehydes and ketoaldehdyes, for example, glyoxal, phenylglyoxal or pyruvic aldehyde, can optionally be used.

### Promoter Catalyst System—Ion Exchange Resin

The promoter catalyst system of the present disclosure comprises an ion exchange resin catalyst and a promoter. In one aspect, the ion exchange resin can comprise any ion exchange resin suitable for use in the catalyst system of the present invention. In another aspect, the ion exchange resin comprises a cross-linked cationic exchange resin. In another aspect, the ion exchange resin comprises a cross-linked sulfonated ion exchange resin having a plurality of sulfonic acid sites. In yet another aspect, the ion exchange resin is acidic or strongly acidic. In one aspect, at least a portion of the ion exchange resin comprises sodium polystyrene sulfonate. In still other aspects, the ion exchange resin can comprise a monodispersed resin, a polydispersed resin, or a combination thereof.

The specific chemistry of an ion exchange resin or any one or more polymer materials that form a part of an ion exchange resin can vary, and one of skill in the art, in possession of this disclosure, could readily select an appropriate ion exchange resin. In one aspect, the ion exchange resin comprises polystyrene or a derivatized polystyrene. In another aspect, the ion exchange resin comprises a polysiloxane or derivatized polysiloxane. It should also be understood that the catalyst system can, in one aspect, comprise multiple ion exchange resins of the same or varying composition, acidity, and/or degree of cross-linking.

In one aspect, the ion exchange resin can be cross-linked with the same or a different polymer material. In various aspects, the degree of cross-linking is from about 1 percent to about 8 percent, for example, about 1, 2, 3, 4, 5, 6, 7, 8, percent; from about 1 percent to about 4 percent, for example, about 1, 1.2, 1.4, 1.6, 1.8, 2, 2.2, 2.4, 2.6, 2.8, 3, 3.2, 3.4, 3.6, 3.8, or 4 percent; or from about 1.5 percent to about 2.5 percent, for example, about 1.5, 1.6, 1.7, 1.8, 1.9, 2, 2.1, 2.2, 2.3, 2.4, or 2.5 percent. In other aspects, the degree of cross-linking can be less than 1 percent or greater than 8 percent, and the present invention is not intended to be limited to any particular degree of cross-linking recited here. In a specific aspect, the degree of cross-linking is about 2 percent. In another aspect, the ion exchange resin is not cross-linked. While not wishing to be bound by theory, cross-linking of an ion exchange resin is not necessary, but can provide additional stability to the resin and the resulting catalyst system.

In one aspect, the ion exchange resin can be cross-linked using any conventional cross-linking agents, such as, for example, polycyclic aromatic divinyl monomers, divinyl benzene, divinyl toluene, divinyl biphenyl monomers, or combinations thereof.

In other aspects, the ion exchange resin comprises a plurality of acid sites, and has, before modification, at least about 3, at least about 3.5, at least about 4, at least about 5, or more acid milliequivalents per gram (meq/g) when dry. In a specific aspect, the ion exchange resin, before modification, has at least about 3.5 acid milliequivalents per gram when dry. In various aspects, any of the plurality of acid sites on an ion exchange resin can comprise a sulfonic acid functionality, which upon deprotonation produces a sulfonate anion functionality, a phosphonic acid functionality, which upon deprotonation produces a phosphonate anion functionality, or a carboxylic acid functionality, which upon deprotonation produces a carboxylate anion functionality.

Exemplary ion exchange resins can include, but are not limited to, DIAION® SK104, DIAION® SK1B, DIAION® PK208, DIAION® PK212 and DIAION® PK216 (manufactured by Mitsubishi Chemical Industries, Limited), A-121, A-232, and A-131, (manufactured by Rohm & Haas), T-38, T-66 and T-3825 (manufactured by Thermax), LEWATIT® K1131, LEWATIT® K1221 (manufactured by Lanxess), DOWEX® 50W2X, DOWEX® 50W4X, DOWEX® 50W8X resins (manufactured by Dow Chemical), Indion 180, Indion 225 (manufactured by Ion Exchange India Limited), and PUROLITE® CT-222 and PUROLITE® CT-122 (manufactured by Purolite).

### Promoter Catalyst System—Promoter

The promoter of the attached promoter catalyst system of the present invention can comprise any promoter species suitable for use in the various methods described herein, and that can provide a desired high-purity product.

In one aspect, the promoter of the present invention can comprise pyridyl ethylmercapton (PEM). In another aspect, the promoter of the present invention can comprise dimethyl thiazolidine (DMT). In other aspects, the promoter of the present invention can comprise derivatives and/or analogues of pyridyl ethylmercapton, dimethyl thiazolidine, or a combination thereof. In another aspect the promoter of the present invention can comprise other promoter species not specifically recited herein. In another aspect, the promoter of the present invention can be represented by the formula below:

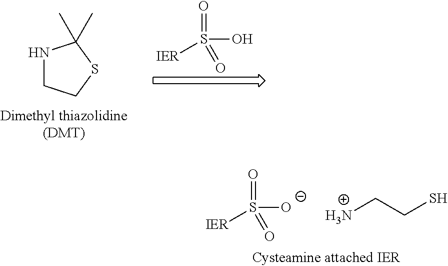

wherein the dimethyl thiazolidine is combined with an ion exchange resin so as to provide a cysteamine attached ion exchange resin.

In one aspect, the promoter can be contacted with the ion exchange resin so as to neutralize at least a portion of the available acid sites on the ion exchange resin, and attach thereto. In various aspects, the ion exchange resin is modified by neutralizing from about 10% to about 40% of the available acid sites with the promoter; or from about 18% to about 25% of the available acid sites with the promoter. In another aspect, the promoter is bound to from about 18% to about 25%, for example, about 18, 19, 20, 21, 22, 23, 24, or 25% of the acid sites on the ion exchange resin. In another aspect, the promoter is bound to from about 20% to about 24% of the acid sites on the ion exchange resin. In still another aspect, the promoter is bound to about 22% of the acid sites of the ion exchange resin.

In an exemplary process, the promoter is combined with a solvent to form a mixture. The mixture may further comprise an acid to improve solubility of the promoter. In one aspect, the amount of acid can be sufficient to solubilize the promoter but not enough to impede modification of the ion exchange resin. In one aspect, the amount of acid is typically less than or equal to about 1 equivalent; or less than or equal to about 0.25 equivalents, based on the number of moles of the promoter. Exemplary acids include, but are not limited to, hydrochloric acid (HCl), p-toluenesulfonic acid, trifluorocacetic acid, and acetic acid. In such an aspect, the mixture can be contacted with the ion exchange resin resulting in an ionic linkage between the promoter cation and anion (deprotonated acid site) of the ion exchange resin. Formation of the ionic linkage neutralizes the acid site.

The degree of neutralization may be determined in a number of ways. In one aspect, the modified ion exchange resin catalyst can be titrated to determine the amount of remaining acid sites.

Following modification (neutralization), the modified ion exchange resin catalyst can optionally be rinsed with a continuous flow of phenol to remove any remaining amounts of solvent from the modification. Alternatively, if acid was used to improve the solubility of the promoter, the modified ion exchange resin can optionally be rinsed with deionized water prior to rinsing with phenol. In one aspect, removing substantially all of the water is herein defined as removing greater than or equal to about 75%, greater than or equal to about 80%, or greater than or equal to about 85%, based on the total amount of water initially employed.

In one aspect, at least a portion of the promoter is ionically bound to the available acid sites of the ion exchange resin. In another aspect, all or substantially all of the promoter is ionically bound to acid sites of the ion exchange resin. In another aspect, at least a portion of the promoter is covalently bound to at least a portion of the ion exchange resin. In still another aspect, all or substantially all of the promoter is at least covalently bound to the ion exchange resin. In yet another aspect, the degree of attachment or binding between a promoter and an ion exchange resin can vary, such as, for example, covalent binding, ionic binding, and/or other interactions or attraction forces, and the present invention is not intended to be limited to any particular degree of attachment.

It should be noted that the methods described herein comprise multiple optional steps, and that no specific or required order of steps is intended, except where such an order would not be functional. One of skill in the art could readily determine which optional steps to utilize and in which order any reaction steps should be performed so as to produce a desired result.

### Condensation Reaction

In one aspect, the reactants, phenol or optionally purified phenol, and at least one of a ketone or aldehyde, can be fed into a reactor vessel and contacted. In another aspect, the reactants, once in a reactor vessel, can be mixed using, for example, a static mixer. After contacting and/or mixing, the reactor feed comprising the reactants can be cooled to a predetermined temperature using, for example, a plate heat exchanger. In one aspect, use of the attached promoter catalyst system can allow for a reduction in the amount of acetone in the reactor feed stream, for example, from about 9.5 wt. % to about 5 wt. %. In such an aspect, the reactor effluent can have a lower solids content and a higher quantity of phenol.

A bed of the attached promoter ion exchange resin catalyst system, such as, for example, a cross-linked ion exchange resin catalyst with attached dimethyl thiazolidine promoter) can be disposed in the reaction vessel, such that the reactants flow through the bed. In one aspect, the reaction vessel and bed can be oriented such that the reactants flow downward (e.g., gravity-fed) through the catalyst bed.

In various aspects, the reaction can be controlled to a predetermined temperature, for example, about 55° C. or 65° C. Variations in temperature can affect the rate of reaction and rate of isomerization of any produced BPA. Other temperatures not recited herein can be utilized, and one of skill in the art could readily determine an appropriate temperature at which to conduct a particular condensation reaction.

In one aspect, the reactor can be capable of converting at least about 90% of the acetone, if present, in the reactor feed. In other aspects, the reactor can be capable of converting at least about 92%, 94%, 96%, 98%, or more of the acetone, if present, in the reactor feed. In another aspect, the reactor and attached promoter catalyst system can be capable of producing the p,p-BPA isomer with a selectivity of at least about 90%. In other aspects, the reactor and attached promoter catalyst system can be capable of producing the p,p-BPA isomer with a selectivity of at least about 90%, at least about 92%, at least about 93%, at least about 95%, at least about 97%, or more.

Once passed through the catalyst bed, the reactor effluent can optionally be subjected to a separate ion exchange resin bed to remove any undesired materials, such as, for example, oligomers, from the process stream. In one aspect, the reactor effluent is subjected to a separate ion exchange resin bed to remove any undesired materials. In another aspect, the reactor effluent is not subjected to a separate ion exchange resin bed.

### Water Removal

After reaction, the effluent stream can optionally be subjected to a water removal step to remove residual water. In various aspects, a water removal step, if performed, can comprise one or multiple columns positioned in sequence.

In one aspect, the reactor effluent stream can comprise water, acetone, phenol, toluene and/or other aromatic solvents, such as, for example benzene and xylene. In one aspect, the effluent (i.e., vapor) can be subjected to a water cooled process condenser. In another aspect, the vent gas from such process condenser can be conveyed to a brine vent condenser, cooled by, for example, chilled brine at a temperature of about 8° C. It should be noted that the specific parameters (e.g., temperature) described herein are intended to be exemplary and non-limiting, and that one of skill in the art, in possession of this disclosure, could readily determine appropriate experimental conditions for a given process setup.

In another aspect, an inert gas, such as, for example, nitrogen, can be introduced in the condenser units so as to at least partially prevent the condensation of an aromatic solvent, such as, for example, toluene, that can be present in the effluent stream.

After passing through a water removal step, the reactor effluent (i.e., dehydrated reactor effluent) can have water content of less than about 0.5 wt. %, less than about 0.4 wt. %, less than about 0.3 wt. %, less than about 0.2 wt. %, or less than about 0.1 wt. %. In one aspect, the dehydrated reactor effluent has a water content of less than about 0.2 wt. %. In another aspect, the dehydrated reactor effluent has a water content of about 0.1 wt. %.

### Phenol Recovery

To accommodate the change in reactor effluent from the use of an attached promoter ion exchange resin catalyst system, the reactor effluent stream, after optionally passing through a separate ion exchange resin bed and/or water removal step, can, in one aspect, comprise a phenol flash (i.e., phenol recovery) unit.

In various aspects, a phenol flash unit can comprise a single column or a plurality of individual columns. In various aspects, any combination of columns or order of columns can be utilized. In one aspect, a phenol flash unit comprises three individual columns: a phenol flash column, an upper phenol column, and a lower phenol column. In another aspect, any one or multiple columns can be removed from a process. It should be noted that the terms “upper” and “lower” are not intended to require a particular orientation, and the respective columns can be positioned in any geometric arrangement appropriate for a particular process.

In one aspect, the phenol flash column removes all or a portion of phenol reactants from the effluent stream. In a specific aspect, the reactor effluent, such as, for example, the dehydrated reactor effluent, can be heated prior to entering the phenol flash column. In another aspect, the phenol flash column can be operated at an elevated temperature and/or under vacuum, such as for example, about 750 mbar.

In one aspect, flashed phenol can be condensed after removal from the effluent stream. In another aspect, flashed phenol can be at least partially condensed using the feed stream to the flash column as a cooling medium. In such an aspect, at least a portion of the energy expended to flash the phenol can be recovered. Any remaining phenol vapors, if present, can be recovered by, for example, a vacuum system.

In one aspect, the effluent stream can be first subjected to a phenol flash column. The effluent from the phenol flash column can then be subjected to an upper phenol column, if present, and then to a lower phenol column.

In one aspect, the effluent from the phenol flash unit, for example, after passing through a phenol flash column, an upper phenol column, and a lower phenol column, can have a free phenol content of less than about 1.0 wt. %, less than about 0.75 wt. %, or less than about 0.5 wt. %. In a specific aspect, the effluent from the phenol flash unit has a free phenol content of less than about 0.5 wt. %.

It should be noted that, at any point in the process, an effluent stream can optionally be redirected back through one or more units and/or columns of the reaction process so as to further react and/or purify the effluent. One of skill in the art, in possession of this disclosure, could readily determine when any such recirculation loop should be employed so as to produce a desired product.

### Solvent Crystallization

In one aspect, the reactor effluent, after being subjected to the phenol flash unit, can be fed to a solvent crystallization unit. In one aspect, a solvent crystallization unit can be used to remove BPA byproducts, such as, for example, o,p-BPA, chromans, BPX 1 and/or 2, cyclic dimers (CD1 and/or 2), linear dimers (LD2 and/or 2), or a combination thereof. Structures of these BPA impurities are shown in e.g. Nowakowska et al., Polish J. Appl. Chem., XI(3), 247-254, 1996. In one aspect, the solvent crystallization unit can remove at least a portion of the BPA byproducts from the effluent stream. In one aspect, the solubility of one or more individual components in an effluent stream can be known or determined. In another aspect, such solubility data can be used by one of skill in the art to optimize the crystallization parameters so as to provide an improved product.

Properties of Produced Bisphenol-A and BPA Polycarbonate

Reaction products of the various methods of the present invention can, in various aspects, exhibit higher purity levels than are attainable using conventional manufacturing processes. In one aspect, the use of an attached promoter ion exchange resin catalyst system, such as, for example, a dimethyl thiazolidine catalyst system, can provide a resulting BPA product that has no or substantially no inorganic or sulfur impurities. Moreover, the combination of an attached promoter catalyst system and a solvent crystallization step can provide products having high purity levels and that are suitable for use in the manufacture of food contact grade materials and materials for demanding optical applications. For example, BPA produced using the methods of the present invention can be utilized in the manufacture of food-grade polycarbonate products.

In one aspect, BPA synthesized using the methods of the present invention can be useful in producing polycarbonate having enhanced optical properties as compared to a conventional polycarbonate produced from a conventional BPA material. In one aspect, BPA prepared from the methods of the present invention can produce a polycarbonate having good impact strength (ductility). Conventional polycarbonates can age upon exposure to heat, light, and/or over time, resulting in reduced light transmission and color changes within the material

Conventional bulk promoter catalyst systems that utilize resin catalyst systems with sulfonic acid groups and 3 MPA promoters can leave up to about 20 ppm sulfur or more in the resulting BPA, even after purification. In one aspect, the methods described herein can provide a BPA having less than about 10 ppm, less than about 5 ppm, less than about 4 ppm, less than about 3 ppm, less than about 2 ppm, or less than about 1 ppm sulfur, for example, as measured by combustion and/or coulometric methods. In a specific aspect, the methods described herein can provide a BPA having less than about 2 ppm sulfur. In another aspect, the methods described herein can provide a BPA that is free of or substantially free of sulfur.

In another aspect, the improved purity, for example, reduced sulfur and organic contaminants, of BPA produced using the methods described herein can result in polycarbonate materials having improved color properties. In one aspect, polycarbonate produced from BPA prepared by the methods of the present disclosure can exhibit reduced color, for example, yellowness, as compared to conventional polycarbonate materials, even after aging at elevated temperatures. In one aspect, a polycarbonate produced from BPA prepared by the methods of the present disclosure can exhibit surprisingly low color after aging for 2,000 hours at about 130° C.

In another aspect, the BPA produced by the methods described herein, or a polycarbonate, for example, a BPA polycarbonate, prepared therefrom, can comprise less than or equal to about 150 ppm of free hydroxyl groups, for example, about 150, 125, 100, 75, 50 ppm, or less free hydroxyl groups.

In one aspect, the yellowness index (YI), as measured by ASTM D1925, of a 2.5 mm thick polycarbonate plaque formed from a bisphenol-A monomer using the methods of the present disclosure, can be less than about 1.6, for example, less than about 1.6, less than about 1.5, less than about 1.4, or less than about 1.3. In a specific aspect, a 2.5 mm thick polycarbonate plaque can have a yellowness index of less than about 1.5. In another aspect, a 2.5 mm thick polycarbonate plaque can have a yellowness index of less than about 1.3. In another aspect, the yellowness index (YI), as measured by ASTM D1925, of a mm thick polycarbonate plaque formed from a bisphenol-A monomer using the methods of the present disclosure, after heat aging for 2,000 hours at about 130° C., can be less than about 10, for example, less than about 10, less than about 9, less than about 8, less than about 7, less than about 6, or less than about 5. In a specific aspect, the yellowness index of a 2.5 mm thick polycarbonate plaque, after heat-aging, can be less than about 10. In another aspect, the yellowness index of a 2.5 mm thick polycarbonate plaque, after heat-aging, can be less than about 7. In another aspect, the yellowness index of a 2.5 mm thick polycarbonate plaque, after heat-aging, can be less than about 5. In another aspect, the yellowness index of a 2.5 mm thick polycarbonate plaque, after heat-aging, can be less than about 2.

In another aspect, BPA polycarbonate produced from the methods described herein can have a purity level suitable for use in optical applications requiring high transmission and low color, wherein the BPA polycarbonate is manufactured from bisphenol-A prepared by contacting at least two chemical reagents with an attached promoter ion exchange resin catalyst system to produce an effluent, and then subjecting the effluent to a solvent crystallization step.

In one aspect, BPA polycarbonate manufactured from bisphenol-A prepared by the methods described herein can have a transmission of at least about 90%, for example, about 90%, 92%, 94%, 96%, 98%, or more, at a thickness of 2.5 mm, as measured by ASTM D1003-00. In other aspects, a BPA polycarbonate, as described herein, can have no or substantially no sulfur impurities. In another aspect, a BPA polycarbonate, as described herein, can have an organic purity of at least about 99.5%. In another aspect, a BPA polycarbonate, as described herein, can have less than or equal to about 150 ppm free hydroxyl groups. In still other aspects, a BPA polycarbonate, as described herein, can have a sulfur concentration of less than about 5 ppm or less than about 2 ppm.

In another aspect, the invention can comprise an article comprising a BPA polycarbonate, for example, a polycarbonate manufactured from BPA produced by the methods described herein. In other aspects, such an article can be selected from at least one of the following: a light guide, a light guide panel, a lens, a cover, a sheet, a bulb, and a film. In a specific aspect, the article can comprise a LED lens. In another aspect, the article can comprise at least one of the following: a portion of a roof, a portion of a greenhouse, and a portion of a veranda.

In other aspects, BPA prepared by the methods described herein can be used to produce polycarbonate resins and/or polycarbonate copolymer materials, for example a polyester-polycarbonate copolymer, a polysiloxane-polycarbonate copolymer, an alkylene terephthalate-polycarbonate copolymer, or a combination thereof. In other aspects, BPA prepared by the methods described herein can be used to produce other polycarbonate copolymers not specifically recited herein, and the present invention is not intended to be limited to any particular polycarbonate and/or polycarbonate copolymer material.

In one aspect, the bisphenol-A, polycarbonate, and article of the present disclosure can comprise any combination of components, purities, and properties described herein, including various aspects wherein any individual component, purity, and/or property, such as, for example, sulfur level, yellowness index, organic purity, and/or transmission can be either included or excluded from the composition. Thus, combinations wherein comprising any one or more components, purities, and/or properties, but excluding other components, purities, and/or properties recited herein are contemplated.

### EXAMPLES OF THE EMBODIMENTS

In one embodiment, a bisphenol-A monomer has an organic purity of at least about 99.5 wt. % and a sulfur concentration of less than about 5 ppm, wherein when formed into a polycarbonate resin and molded into a 2.5 mm plaque, exhibits a color (YI) of less than about 1.5.

In another embodiment, a BPA polycarbonate has a purity level suitable for use in optical applications requiring high transmission and low color, wherein the BPA polycarbonate is manufactured from bisphenol-A prepared by contacting at least two chemical reagents with an attached promoter ion exchange resin catalyst system to produce an effluent, and then subjecting the effluent to a solvent crystallization step

In the various embodiments, (i) the bisphenol-A monomer has a sulfur concentration of less than about 2 ppm; and/or (ii) the bisphenol-A monomer, when formed into a polycarbonate resin and molded into a 2.5 mm plaque, exhibits a yellowness index (YI) of less than about 1.3, as measured by ASTM D1925; and/or (iii) the bisphenol-A monomer. when formed into a polycarbonate resin and molded into a 2.5 mm plaque, exhibits a yellowness index (YI), as measured by ASTM D1925, of less than about 10 after heat aging for 2,000 hours at about 130° C.; and/or (iv) the bisphenol-A monomer, when formed into a polycarbonate resin and molded into a 2.5 mm plaque, exhibits a yellowness index, as measured by ASTM D1925, (YI) of less than about 7 after heat aging for 2,000 hours at about 130° C.; and/or (v) the bisphenol-A monomer, when formed into a polycarbonate resin, has a transmission level of at least about 90% at a 2.5 mm thickness, as measured by ASTM D1003-00; and/or (vi) the bisphenol-A monomer, when formed into a polycarbonate resin, has less than or equal to about 150 ppm free hydroxyl groups; and/or (vii) a polycarbonate or copolymer comprises polycarbonate prepared from any of the above-described bisphenol-A embodiments; and/or (viii) the polycarbonate or copolymer comprises one or more of a polyester-polycarbonate copolymer, a polysiloxane-polycarbonate copolymer, an alkylene terephthalate-polycarbonate copolymer, or a combination thereof; and/or (ix) the polycarbonate or copolymer has a yellowness index (YI) of less than about 1.3, as measured by ASTM D1925, when formed into a 2.5 mm thick plaque; and/or (x) the polycarbonate or copolymer has a yellowness index (YI) of less than about 10, as measured by ASTM D1925, when formed into a 2.5 mm thick plaque and heat aged for 2,000 hours at about 130° C.; and/or (xi) the polycarbonate has no or substantially no sulfur impurities; and/or (xii) the polycarbonate has an organic purity of at least about 99.5%; and/or (xiii) the polycarbonate has less than or equal to about 150 ppm free hydroxyl groups; and/or (xiv) the polycarbonate has a transmission of at least about 90% at 2.5 mm thickness, as measured by ASTM D1003-00; and/or (xv) the polycarbonate has a sulfur level of less than about 5 ppm; and/or (xvi) the polycarbonate has a sulfur level of less than about 2 ppm; and/or (xvii) the polycarbonate has a yellowness index (YI) at 2.5 mm thickness, as measured by ASTM D1925, of less than about 1.5; and/or (xviii) the polycarbonate has a yellowness index (YI) at mm thickness, as measured by ASTM D1925, of less than about 10 after heat aging for 2,000 hours at about 130° C.; and/or (xix) the polycarbonate has a yellowness index (YI), at mm thickness, as measured by ASTM D1925, of less than about 7 after heat aging for 2,000 hours at about 130° C.; and/or (xx) the polycarbonate has a yellowness index (YI), at mm thickness, as measured by ASTM D1925, of less than about 2 after heat aging for 2,000 hours at about 130° C.; and/or (xxi) the BPA polycarbonate is an interfacially polymerized polycarbonate; and/or (xxii) the polycarbonate comprises a flame retardant; and/or (xxiii) the polycarbonate further comprises a second polycarbonate derived from bisphenol-A, wherein the second polycarbonate is different than the BPA polycarbonate; and/or (xxiv) the second polycarbonate is selected from wherein the second polycarbonate is selected from at least one of the following: a homopolycarbonate derived from a bisphenol; a copolycarbonate derived from more than on bisphenol; and a copolymer derived from one or more bisphenols and comprising one or more aliphatic ester units or aromatic ester units or siloxane units; and/or (xxv) the polycarbonate further comprises one or more additives selected from at least one of the following: UV stabilizing additives, thermal stabilizing additives, mold release agents, colorants, organic fillers, inorganic fillers, and gamma-stabilizing agents; and/or (xxvi) an article comprises the bisphenol-A and/or the polycarbonate of any one or combination of any the above-described embodiments; and/or (xxvii) the article is selected from at least one of the following: a light guide, a light guide panel, a lens, a cover, a sheet, a bulb, and a film; and/or (xxviii) the article is a LED lens; and/or (xxix) the article comprises at least one of the following: a portion of a roof, a portion of a greenhouse, and a portion of a veranda.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. General Methods

In a first example, phenol and acetone are each fed to a reactor, where they are subsequently mixed using a static mixer. The reactor feeds are cooled using a plate heat exchanger before reaching the reactor vessel. A bed of ion exchange resin having an attached promoter (2% cross-linked ion exchange resin catalyst with DMT attached promoter) is disposed in the reactor such that the reactor feeds flow through the reactor in a downward fashion. Conversion of acetone in the reactor is designed to be at least about 90%, with a p,p-BPA selective of at least about 93%.

After reacting, the effluent stream is transferred to a separate vessel, where it passes through a bed of anion exchange resin to remove any free oligomer. The effluent is then subjected to a water removal step. After the water removal step, the dehydrated effluent stream is subjected to a phenol recovery step where a phenol flash column is used to remove phenol remaining in the effluent stream. The solvent crystallization unit can remove all or substantially all of the BPA byproducts. The remaining effluent stream can then be treated in a solvent recovery system to remove the aromatic solvent(s), such as toluene. The toluene or other aromatic solvent is thus separated from the BPA isomer stream.

### 2. Preparation of BPA

In a second example, BPA samples from different sources (e.g., BPA process catalysts and promoters) were used to produce polycarbonate resins. The polycarbonate resins were produced in a single production facility using an interfacial polymerization process. Molded plaques were then prepared from polycarbonate resin stabilized with 0.05 wt. % IRGAFOS® 168 trisarylphosphite processing stabilizer.

The sulfur content and organic purity of each BPA sample were determined Sulfur measurements were performed using combustion and coulometric method for total sulfur determination. Organic purity was determined using ultraviolet detection after high performance liquid chromatography separation (see HPLC method in Nowakowska et al., Polish J. Appl. Chem., XI(3), 247-254, 1996). The organic purity is defined as 100 wt. % less the sum of known and unknown impurities detected via ultraviolet radiation at 280 nm.

The color of each 2.5 nm plaque was determined, according to ASTM D1925, after molding (YI0), as well as after heat aging for 2,000 hours at 130° C. (YI, 2000 hrs 130 C). Table 1, below illustrates the color, purity, and sulfur concentration for each sample. Samples prepared using BPA from a conventional bulk promoter system, wherein an ion exchange resin with sulfonic acid groups is used in combination with a 3 MPA promoter, as identified as “BP” in the BPA process column. Samples prepared using BPA from a production process using hydrochloric acid as a catalyst are identified as “HCl” in the BPA process column. Samples prepared using BPA from the inventive attached promoter methods described herein are identified as “AP” in the BPA process column.

TABLE 1
Color and Purity Analysis of BPA Materials.

| | | | | | BPA |
|---|---|---|---|---|---|
| | | | | | process |
| | YI | YI (2000 hrs | BPA purity | Sulfur | catalyst/ |
| Example | (—) | 130 C.) | (% w) | (ppm) | promoter |
| Comp. Ex. 1 | 1.88 | 13.40 | 99.44 | 25 | BP |
| Comp. Ex. 2 | 1.85 | 13.07 | 99.52 | 23 | BP |
| Comp. Ex. 3 | 1.96 | 13.37 | 99.45 | 25 | BP |
| Comp. Ex. 4 | 1.78 | 13.20 | 99.52 | 23 | BP |
| Comp. Ex. 5 | 2.01 | 13.61 | 99.44 | 25 | BP |
| Comp. Ex. 6 | 1.59 | 10.29 | 99.54 | 19 | BP |
| Comp. Ex. 7 | 1.65 | 11.74 | 99.47 | 17 | BP |
| Comp. Ex. 8 | 1.47 | 10.92 | 99.45 | 21 | BP |
| Comp. Ex. 9 | 1.80 | 10.61 | 99.39 | 23 | BP |
| Comp. Ex. 10 | 1.57 | 14.33 | 99.50 | 18 | BP |
| Comp. Ex. 11 | 1.49 | 12.40 | 99.51 | 16 | BP |
| Comp. Ex. 12 | 1.39 | 10.01 | 99.57 | 18 | BP |
| Comp. Ex. 13 | 1.65 | 11.72 | 99.47 | 21 | BP |
| Comp. Ex. 14 | 1.69 | 10.76 | 99.61 | <2 | HCl |
| Comp. Ex. 15 | 1.66 | 10.45 | 99.62 | <2 | HCl |
| Ex. 16 | 1.20 | 6.79 | 99.53 | <2 | AP |
| Ex. 17 | 1.35 | 6.24 | 99.54 | <2 | AP |
| Ex. 18 | 1.26 | 6.72 | 99.54 | <2 | AP |
| Ex. 19 | 1.29 | 7.63 | 99.57 | <2 | AP |
| Ex. 20 | 1.27 | 8.66 | 99.50 | <2 | AP |
| Ex. 21 | 1.31 | 8.71 | 99.56 | <2 | AP |
| Ex. 22 | 1.25 | 4.93 | 99.78 | <2 | AP |
| Ex. 23 | 1.39 | 8.92 | 99.55 | <2 | AP |
| Ex. 24 | 1.42 | 8.04 | 99.57 | <2 | AP |
| Ex. 25 | 1.33 | 5.38 | 99.75 | <2 | AP |
| Ex. 26 | 1.36 | 4.57 | 99.78 | <2 | AP |

The BPA prepared using conventional bulk promoter systems has about 20 ppm sulfur, even after purification of the monomer. The BPA prepared using HCl exhibited a sulfur level of less than about 2 ppm. Similarly, the BPA prepared from the attached

prompter systems described herein exhibited less than about 2 ppm sulfur (i.e., a level below the detection limit of the measurement equipment).

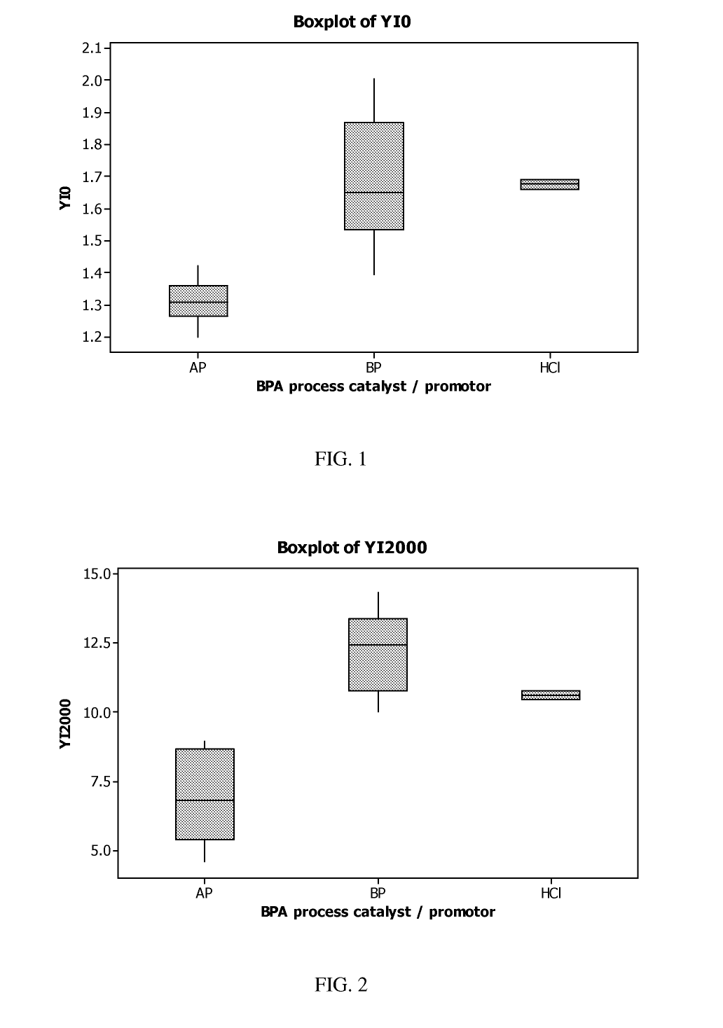

As detailed in Table 1, the color (i.e., yellowing) of polycarbonate plaques prepared from each of the BPA samples was measured. Polycarbonate resins prepared from conventional bulk promoter (BP) and HCl derived BPA exhibited substantially higher yellowing than resins prepared from attached promoter (AP) derived BPA, both for as-molded plaques and heat-aged plaques. Graphical summaries of the color measurements (yellowness) after molding and after heat aging for 2,000 hours at 130° C. are illustrated in FIGS. 1 and 2, respectively. For both the as-molded and heat-aged plaques, polycarbonate resins produced from BPA prepared by the attached promoter methods of the present disclosure exhibited significantly less yellowing, as compared to polycarbonate resins produced from HCl and conventional bulk promoter (BP) BPA.

While BPA prepared from HCl can exhibit good purity and low sulfur levels, the polycarbonate that is made from it does not have the reduced yellowing benefit as the polycarbonate that is obtained from BPA prepared with the attached promoter methods described in the present disclosure. BPA prepared from conventional bulk promoter (BP) systems exhibits both higher sulfur content and yellowing (in the derived polycarbonate), as compared to BPA prepared with the attached promoter methods of the present disclosure.

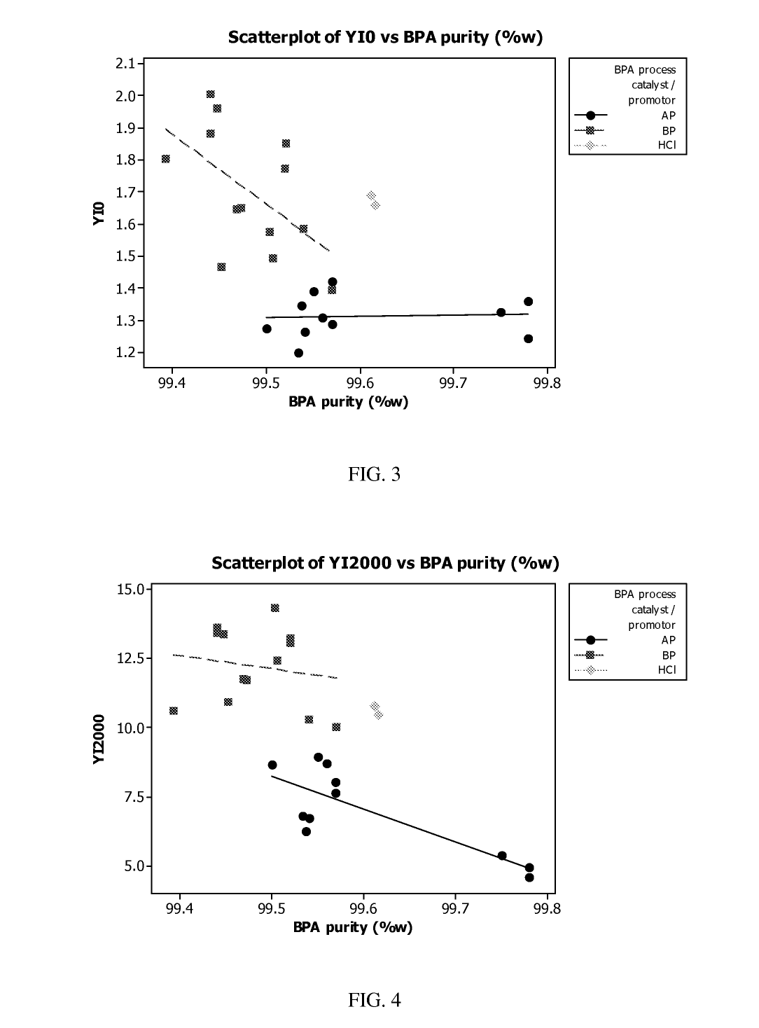

Plots of BPA purity versus color (i.e., yellowing) for as-molded plaques and for heat-aged plaques, are illustrated in FIGS. 3 and 4.

Statistical analysis (ANOVA) indicates a significant difference (95% confidence) between the AP derived samples and the other materials for both starting color as well as color after heat aging. Comparing inventive examples 16-26 with comparative examples 14 and 15 shows that this improved color is not just related to the sulfur content in the resin, which is one of the differences when comparing AP and BP derived materials. The overall organic purity itself is not the only factor in determining color and color stability either as shown in the more detailed graphs (FIGS. 3 & 4) below. Although a higher organic monomer purity appears to lead to lower yellowing for the BP derived samples, the AP derived samples clearly outperform the BP materials at a given purity of e.g. 99.55%.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A bisphenol-A monomer having an organic purity of at least about 99.5 wt. % and a sulfur concentration of less than about 5 ppm, wherein when formed into a polycarbonate resin and molded into a 2.5 mm plaque, exhibits a color (YI) of less than about 1.5.

2. The bisphenol-A monomer of claim 1, having a sulfur concentration of less than about 2 ppm.

3. The bisphenol-A monomer of claim 1, wherein when formed into a polycarbonate resin and molded into a 2.5 mm plaque, exhibits a yellowness index (YI) of less than about 1.3, as measured by ASTM D1925.

4. The bisphenol-A monomer of claim 1, wherein when formed into a polycarbonate resin and molded into a 2.5 mm plaque, exhibits a yellowness index (YI), as measured by ASTM D1925, of less than about 10 after heat aging for 2,000 hours at about 130° C.

5. The bisphenol-A monomer of claim 4, wherein when formed into a polycarbonate resin and molded into a 2.5 mm plaque, exhibits a yellowness index, as measured by ASTM D1925, (YI) of less than about 7 after heat aging for 2,000 hours at about 130° C.

6. The bisphenol-A monomer of claim 1, wherein when formed into a polycarbonate resin, has a transmission level of at least about 90% at a 2.5 mm thickness, as measured by ASTM D1003-00.

7. The bisphenol-A monomer of claim 1, wherein when formed into a polycarbonate resin, has less than or equal to about 150 ppm free hydroxyl groups.

8. A polycarbonate or copolymer comprising polycarbonate prepared from the bisphenol-A of claim 1.

9. The polycarbonate or copolymer of claim 8, comprising one or more of a polyester-polycarbonate copolymer, a polysiloxane-polycarbonate copolymer, an alkylene terephthalate-polycarbonate copolymer, or a combination thereof.

10. The polycarbonate or copolymer of claim 8, having a yellowness index (YI) of less than about 1.3, as measured by ASTM D1925, when formed into a 2.5 mm thick plaque.

11. The polycarbonate or copolymer of claim 8, having a yellowness index (YI) of less than about 10, as measured by ASTM D1925, when formed into a 2.5 mm thick plaque and heat aged for 2,000 hours at about 130° C.

12. A BPA polycarbonate having a purity level suitable for use in optical applications requiring high transmission and low color, wherein the BPA polycarbonate is manufactured from bisphenol-A prepared by contacting at least two chemical reagents with an attached promoter ion exchange resin catalyst system to produce an effluent, and then subjecting the effluent to a solvent crystallization step.

13. The BPA polycarbonate of claim 12, having no or substantially no sulfur impurities.

14. The BPA polycarbonate of claim 12, having an organic purity of at least about 99.5%.

15. The BPA polycarbonate of claim 12, having less than or equal to about 150 ppm free hydroxyl groups.

16. The BPA polycarbonate of claim 12, having a transmission of at least about 90% at 2.5 mm thickness, as measured by ASTM D1003-00.

17. The BPA polycarbonate of claim 12, having a sulfur level of less than about 5 ppm.

18. The BPA polycarbonate of claim 17, having a sulfur level of less than about 2 ppm.

19. The BPA polycarbonate of claim 12, having a yellowness index (YI) at 2.5 mm thickness, as measured by ASTM D1925, of less than about 1.5.

20. The BPA polycarbonate of claim 12, having a yellowness index (YI) at 2.5 mm thickness, as measured by ASTM D1925, of less than about 10 after heat aging for 2,000 hours at about 130° C.

21. The BPA polycarbonate of claim 20, having a yellowness index (YI), at 2.5 mm thickness, as measured by ASTM D1925, of less than about 7 after heat aging for 2,000 hours at about 130° C.

22. The BPA polycarbonate of claim 21, having a yellowness index (YI), at 2.5 mm thickness, as measured by ASTM D1925, of less than about 2 after heat aging for 2,000 hours at about 130° C.

23. The BPA polycarbonate of claim 12, wherein the BPA polycarbonate is an interfacially polymerized polycarbonate.

24. The BPA polycarbonate of claim 12, comprising a flame retardant.

25. The BPA polycarbonate of claim 12, further comprising a second polycarbonate derived from bisphenol-A, wherein the second polycarbonate is different than the BPA polycarbonate.

26. The BPA polycarbonate of claim 25, wherein the second polycarbonate is selected from wherein the second polycarbonate is selected from at least one of the following: a homopolycarbonate derived from a bisphenol; a copolycarbonate derived from more than on bisphenol; and a copolymer derived from one or more bisphenols and comprising one or more aliphatic ester units or aromatic ester units or siloxane units.

27. The BPA polycarbonate of claim 8, further comprising one or more additives selected from at least one of the following: UV stabilizing additives, thermal stabilizing additives, mold release agents, colorants, organic fillers, inorganic fillers, and gamma-stabilizing agents.

28. An article comprising the bisphenol A of claim 1 or the polycarbonate of claim 8.

29. The article of claim 28, wherein the article is selected from at least one of the following: a light guide, a light guide panel, a lens, a cover, a sheet, a bulb, and a film.

30. The article of claim 28, wherein the article is a LED lens.

31. The article of claim 28, wherein the article comprises at least one of the following: a portion of a roof, a portion of a greenhouse, and a portion of a veranda.
